(19) United States
(12) Patent Application Publication
(10) Pub. No.: US 2012/0150060 A1
(43) Pub. Date: Jun. 14, 2012

(54) METHOD AND SYSTEM TO ESTIMATE IMPEDANCE OF A PSEUDO SENSING VECTOR

(75) Inventor: Xing Pei, Thousand Oaks, CA (US)

(73) Assignee: PACESETTER, INC., Sylmar, CA (US)

(21) Appl. No.: 12/965,143

(22) Filed: Dec. 10, 2010

(51) Int. Cl. A61B 5/05

(52) U.S. Cl. 600/547

(57) ABSTRACT

An implantable medical device (IMD) is provided comprising inputs configured to be coupled to leads having electrodes thereon, wherein combinations of the electrodes are associated with respective active sensing vector. The IMD further comprises an impedance measurement module to collect multiple measured impedances between corresponding combinations of the electrodes. The IMD further includes an impedance derivation module to calculate a derived impedance for at least one pseudo sensing vector based on the measured impedances, wherein the pseudo sensing vector extends to or from at least one pseudo sensing site.

### BACKGROUND OF THE INVENTION

Embodiments of the present invention generally relate to methods and systems that utilize impedance measurements from implantable leads, and more particularly to methods and systems that derive impedance(s) for one or more pseudo sensing vectors based on impedance measurements along active sensing vectors.

Heart failure disease affects many Americans as well as people worldwide, and presents a tremendous economic burden. Part of the cost associated with heart disease relates to hospital admissions due to heart failure events.

Medical devices are implanted in patients to monitor, among other things, electrical activity of a heart and to deliver appropriate electrical and/or drug therapy, as required. Implantable medical devices (“IMDs”) include, for example, pacemakers, cardioverters, defibrillators, implantable cardioverter defibrillators (“ICD”), cardiac resynchronization therapy defibrillators (“CRT”) and the like. The electrical therapy produced by an IMD may include, for example, pacing pulses, cardioverting pulses, anti-tachy pacing (ATP) pulses and/or defibrillator pulses to reverse arrhythmias (e.g., tachycardias and bradycardias) or to stimulate the contraction of cardiac tissue (e.g., cardiac pacing) to return the heart to its normal sinus rhythm.

IMDs may be described as single-chamber or dual-chamber systems. A single-chamber system stimulates and senses in the same chamber of the heart (atrium or ventricle). A dual-chamber system stimulates and/or senses in both chambers of the heart (atrium and ventricle). Dual-chamber systems may typically be programmed to operate in either a dual-chamber mode or a single-chamber mode. Further, IMD systems are known which deliver stimulation pulses at multiple sites. For example, biventricular pacing paces in both ventricles and biatrial pacing paces in both atria. Hence, it is possible, that a heart may be stimulated in all four chambers.

IMDs monitor electrical characteristics of the heart to identify or classify cardiac behavior and to estimate physiological parameters of the heart. For example, known IMDs measure intracardiac and intrathoracic impedance between combinations of electrodes that define sensing sites in the heart and/or chest wall. The electrodes may be located within or proximate to the right atrium (RA), right ventricle (RV), left atrium (LA) and left ventricle (LV). As the left atrium of the heart and associated pulmonary fills with fluid and the lateral atrial pressure (LAP) increases, the impedance measured between electrodes that define a sensing vector that traverses the left atrium may decrease. Conversely, as the fluid level in the left atrium drops, the LAP may decrease and the impedance through the left atrium and associated pulmonary may increase.

Today, various systems have been proposed to monitor and diagnosis heart failure based on measurement of impedance across various paths through the heart and lungs. At least one impedance-based algorithm (PE algorithm) has been proposed to predict and monitor the patient heart failure status. The PE algorithm is used today with implantable cardioverter defibrillators (ICD) and cardiac resynchronization therapy defibrillators (CRT).

However, existing systems and methods may be improved. For example, convention CRT and ICD devices are connected to various combinations of leads, where at least one lead generally has a shocking coil located at an intermediate position within or along the RV. The RV shocking coil is used as a shocking site and as a sensing site. During sensing operations, the RV shocking coil is used in combination with various electrodes to define sensing vectors that extend to/from an intermediate point along the RV chamber. In contrast, other types of IMDs (e.g., pacemakers) are not configured to utilize a “shocking” type coil located at an intermediate position within the RV chamber. Instead, pacemaker leads and other types of leads, that do not have a high voltage intermediate RV electrode, may only have an RV tip electrode and/or an RV ring electrode, both of which are located proximate to the distal end of the RV chamber. CRT and ICD systems have connections for an additional RV shocking electrode, and thus, are able to define an additional sensing vector that is not available through connection with conventional pacemaker-type systems. Given that fewer or different sensing vectors are available in non-ICD/CRT type devices, such systems may exhibit lower sensitivity and/or specificity for certain types of monitoring and diagnosis algorithms.

Moreover, CRT and ICD systems may be initially coupled to a lead that has an RV shocking coil type electrode located at an intermediate position in the RTV chamber. However, over time the lead may experience difficulties with the connection to the RV coil electrode (e.g., a lead fracture and the like). The lead or the RV coil electrode may become compromised such that it is no longer desirable to utilize the RV coil electrode as an actual, active sensing site. Yet certain algorithms implemented in the CRT or ICD may utilize impedance measurements from a sensing vector that extends to/from the RV coil electrode.

A need remains for improved methods and systems for deriving impedance measurements along various sensing vectors, with less dependence on which electrode configurations are available.

### SUMMARY

In accordance with one embodiment, methods and systems are provided to use derived impedance from the existing actual active sensing vectors as a supplement for IMD systems that do not have certain impedance vectors available. Since the derived impedance resembles closely to the real impedance measurement, this can improve the algorithm's sensitivity and specificity, thus provide benefit for the patient. The derived and measured impedance can also be used for self-calibration.

In accordance with one embodiment, methods and systems are provided that use derived impedance from the existing active sensing vectors to supplement for the shock-coil impedance vector in a pacemaker system to improve the HF algorithm's sensitivity and specificity.

In accordance with one embodiment, methods and systems are provided that i) measure impedances of the heart-lung system and obtain the trends; (ii) derive impedances (iii) use the derived impedance for calculation. For example, an ICD system may calculate the difference between the RV coil and the derived impedance. If the different is large than a predefined threshold, the ICD may register a warning that there is a potential system issue.

In accordance with an embodiment, a method is provided for estimating impedance associated with a pseudo sensing vector for an implantable medical device (IMD). The method comprises collecting multiple measured impedances between corresponding multiple combinations of electrodes wherein each combination of electrodes is associated with an active sensing vector. The method also comprises calculating a derived impedance for at least one pseudo sensing vector based on the measured impedances wherein the pseudo sensing vector extends to or from at least one pseudo sensing site.

In accordance with an embodiment an implantable medical device (IMD) is provided comprising inputs configured to be coupled to leads having electrodes thereon, wherein combinations of the electrodes are associated with respective active sensing vector. The IMD further comprises an impedance measurement module to collect multiple measured impedances between corresponding combinations of the electrodes. The IMD further includes an impedance derivation module to calculate a derived impedance for at least one pseudo sensing vector based on the measured impedances, wherein the pseudo sensing vector extends to or from at least one pseudo sensing site.

### BRIEF DESCRIPTION OF THE DRAWINGS

FIG. 1 illustrates a simplified diagram of an implantable medical IMD in electrical communication with leads according to an embodiment.

FIG. 2 illustrates a block diagram of the IMD according to an embodiment.

FIG. 3 illustrates a portion of the active sensing vectors according to an embodiment.

FIG. 4 illustrates a portion of the active sensing vectors according to an embodiment.

FIG. 5 illustrates exemplary pseudo sensing vectors according to an embodiment.

FIG. 6 illustrates a processing sequence in accordance with an embodiment.

### DETAILED DESCRIPTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. The following detailed description is, therefore, not to be taken in a limiting sense, and the scope of the present invention is defined by the appended claims and their equivalents.

Throughout, the terms “a” or “an” shall be used, as is common in patent documents, to include one or more than one. Throughout, the term “or” shall be used to refer to a nonexclusive or, unless otherwise indicated. Throughout, the term “measured impedance” shall refer to intracardiac and/or intrathoracic impedance measurements directly measured from a combination of electrodes positioned within the heart, proximate to the heart and/or within the chest wall. Throughout, the term “derived impedance” shall refer to intracardiac and/or intrathoracic impedance that is not directly measured, but instead is mathematically derived based on measured impedances as described throughout the present specification. Throughout, the term “active sensing vector” shall refer to a path extending between two or more physical, actual electrodes that operate as sensing sites. Throughout, the term “pseudo sensing site” shall refer to a site that may or may not have an electrode but does not have an active sensing electrode. Throughout, the term “pseudo sensing vector” shall refer to a path extending to or from one or more pseudo sensing sites.

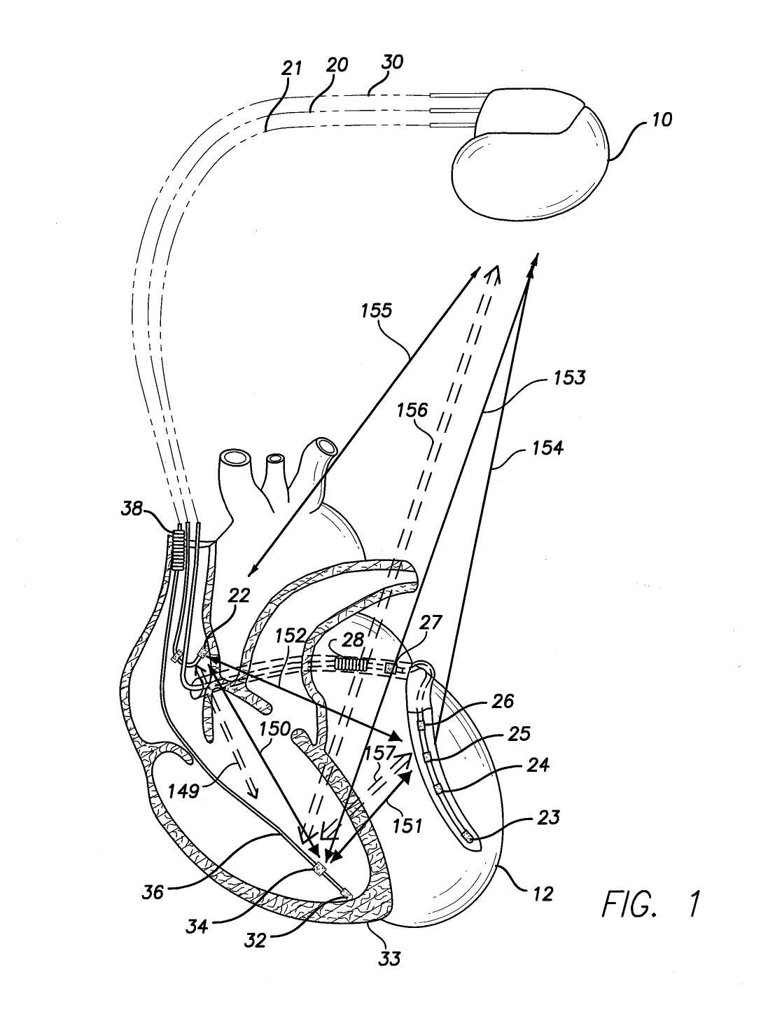

FIG. 1 illustrates a simplified diagram of an implantable medical IMD 10 in electrical communication with three leads 20, 21 and 30 implanted in or proximate a patient's heart 12 for delivering multi-chamber stimulation (e.g. pacing, ATP therapy, high voltage shocks and the like) according to an embodiment. The stimulation may include pacing pulses that are delivered along one or more pacing vectors. Optionally, the stimulation may include ATP pulses or a high voltage shock that is delivered along one or more ATP therapy vectors, cardioverter vectors or defibrillation vectors. The implantable medical IMD 10 may be a pacing device, a pacing apparatus, a cardiac rhythm management device, an implantable cardiac stimulation device, an implantable cardioverter/defibrillator (ICD), a cardiac resynchronization therapy (CRT) device, a monitoring device and the like. The IMD 10 is programmable, by an operator, to set certain operating parameters, as well as therapy-related parameters. The IMD 10 is configured to operate with various configurations of leads. Exemplary lead configurations are shown in the Figures. The IMD 10 is configured to sense various types of information and deliver various types of therapies. For example, the IMD 10 may sense intracardiac electrogram signals, impedances and the like.

In FIG. 1, the IMD 10 is coupled to an RA lead 20 having at least an atrial tip electrode 22, which typically is implanted in the patient's right atrial appendage. The IMD 10 is coupled to an LV lead 21 that includes various electrodes, such as an LV tip electrode 23, intermediate LV electrodes 24-26, and LA electrodes 27-28. The LV lead 21 may sense atrial and ventricular cardiac signals and impedances and deliver left ventricular therapy using the LV tip electrode 23, the intermediate LV electrodes 24-26, and the LA electrodes 27 and 28. Left atrial therapy uses, for example, first and second LA electrodes 27 and 28. The LV and LA electrodes 23-28 may be used as sensing sites, where cardiac signals and/or impedances are sensed, and/or may be used as pacing and/or shock therapy sites. A right ventricular lead 30 may include one or more of an RV tip electrode 32, an RV ring electrode 34, and a superior vena cava (SVC) coil electrode 38 (also known as a RA coil electrode). The right ventricular lead 30 is capable of sensing cardiac signals and/or impedances, and delivering stimulation in the form of pacing and shock therapy to the SVC and/or right ventricle.

Optionally, more or fewer electrodes may be utilized. The LV electrodes may be separated further apart or positioned closer to one another. Optionally, all or a portion of the LV electrodes may be shifted along the LV lead 21 until positioned proximate to the mitral valve, aortic valve, or the left atrial ports to/from the pulmonary veins. The LV lead 21 may be inserted directed into the LV chamber or inserted into a vein or artery extending along the heart wall proximate to the left ventricle. Optionally, the LV lead 21 may be coupled to a patch or mesh net electrode that is secured to or located adjacent to an exterior wall of the left ventricle and/or the left atrium.

Embodiments are described herein, whereby multiple active electrodes are utilized to sense impedance along multiple active sensing vectors in order to measure local impedance information along the active sensing vectors. Impedance measurements collected along the active sensing vectors are utilized to derive impedance for one or more pseudo sensing vector.

The IMD 10 defines active sensing vectors between various combinations of two or more electrodes 22-28, 32, 34 and 38, and the housing of the IMD 10. FIG. 1 illustrates examples of active sensing vectors 150-155, and examples pseudo sensing vectors 149,156 and 157. The active and pseudo sensing vectors 149-157 represent paths (generally a linear path) between at least two points. The IMD 10 obtains one or more impedance measurements along the active sensing vectors 150-155 which extend through a substantial majority of the aortic vessels and the heart 12. An individual measured impedance represents the impedance of the walls of the heart 12, the blood in the heart 12 and any external tissue or muscle through which the corresponding active sensing vector extends.

The active sensing vector 150 extends between the RA electrode 22 and the RV electrode 34. The active sensing vector 151 extends between the RV electrode 34 and the LV electrode 25. The active sensing vector 152 extends between the LV electrode 25 and the RA electrode 22. The active sensing vector 153 extends between the RV electrode 34 and the CAN electrode of the IMD 10. The active sensing vector 154 extends between the LV electrode 25 and the CAN electrode. The active sensing vector 155 extends between the RA electrode 22 and the CAN. Optionally, alternative and/or additional electrodes may be used to form alternative and/or additional active sensing vectors.

The pseudo sensing vector 156 extends between the CAN electrode of the IMD 10 and a virtual electrode site 36. The pseudo sensing vector 149 extends between the RA electrode 122 and a virtual electrode site 36. The pseudo sensing vector 157 extends between the LV electrode 25 and the virtual electrode site 36. There is no “active sensing” electrode at the virtual electrode site 36. In the example of FIG. 1, pseudo sensing vector 156 extends to/from a point in the RV chamber that is located along the lead 30 an intermediate distance from the apex 33 of the RV chamber. It should be recognized that a pacing or shocking electrode may be located at or proximate to the virtual electrode site 36 of the pseudo sensing vector 156, but the pacing or shocking electrode may not be utilized as an active sensing site. Therefore, the pseudo sensing vector 156 may terminate at physical electrodes, but such electrodes are not connected to the sensing circuits in the IMD 10 and do not actively operate as sensing electrodes, nor active sensing sites.

Each LV and RV electrode 22-38 represents a potential sensing site and/or therapy site. When functioning as a sensing site, the corresponding LV and/or RV electrode sense signals that are utilized to obtain impedance measurements. The sensing sites differ based on the type of device and type of detection algorithm utilized.

For example, in a CRT-D type device, when utilizing the PE algorithm, the device utilizes active sensing vectors that extend between the RV coil electrode and CAN, and between a LV ring electrode and the CAN. In an ICD type device, when utilizing the PE algorithm, the device utilizes active sensing vectors that extend between the RV coil electrode and the CAN and between the RV ring electrode and the CAN. In a CRT-P type device, when utilizing the PE algorithm, the device utilizes active sensing vectors that extend between the LV ring electrode and the CAN, between the RA ring electrode and the CAN, and between the RV ring electrode and CAN. In a pacemaker type device, the device generally utilizes an active sensing vector that extends between the RV ring electrode and the CAN.

The impedance measured along the active sensing vectors 151-155 may be expressed in terms of ohms. Alternatively, the impedance may be expressed as an admittance measurement. The admittance may be inversely related to the impedance. The impedance measured along the active sensing vectors 151-155 may vary based on a variety of factors, including the amount of fluid in one or more chambers of the heart 12 and/or thoracic space. As a result, the impedance measurement may be indicative of LAP. As more blood fills the left atrium and pulmonary veins, the LAP increases. Blood is more electrically conductive than the myocardium of the heart 12. Consequently, as the amount of blood in the left atrium increases, the LAP increases and the impedance measured along the active sensing vector decreases. Conversely, decreasing LAP may result in the impedance measurement increasing as there is less blood in the left atrium and pulmonary veins.

Optionally, impedance measurements along various sensing vectors may be utilized to monitor and characterize pressure and blood flow in other chambers of the heart, such as RA, RV, LA and/or LV pressure and blood flow.

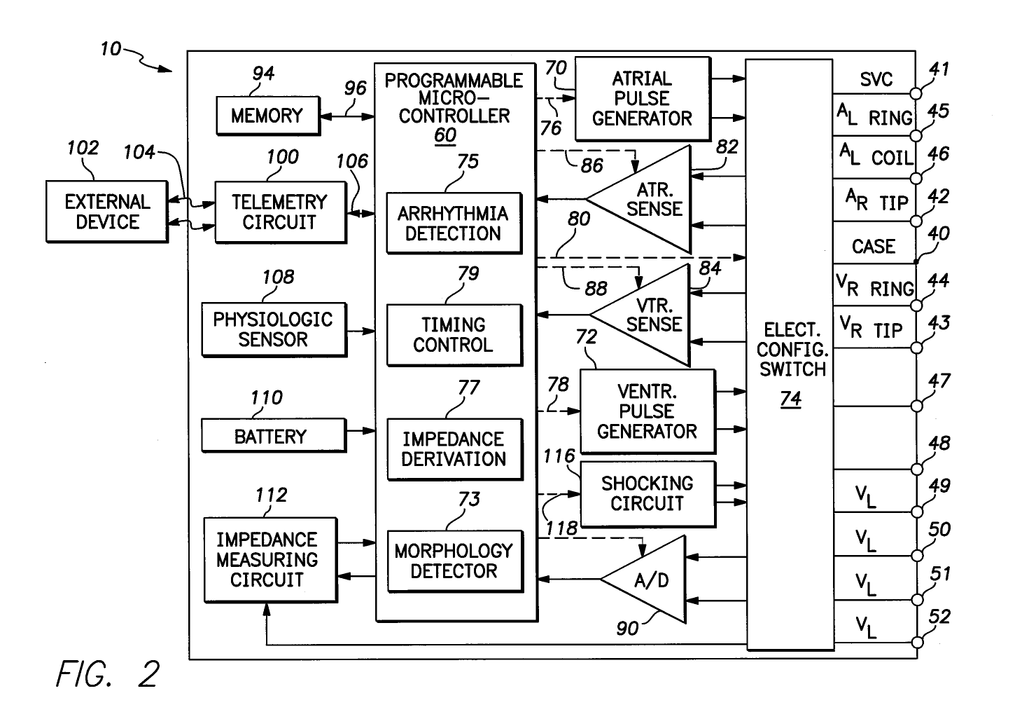

FIG. 2 illustrates a block diagram of the IMD 10, which is capable of treating one or both of fast and slow arrhythmias with stimulation therapy, including cardioversion, defibrillation, and pacing stimulation. While a particular multi-chamber device is shown, this is for illustration purposes only. It is understood that the appropriate circuitry could be duplicated, eliminated or disabled in any desired combination to provide a device capable of simply monitoring impedance and/or cardiac signals, and/or treating the appropriate chamber(s) with cardioversion, defibrillation and pacing stimulation.

The housing 40 for the stimulation IMD 10 is often referred to as the “can”, “case” or “case electrode” and may be programmably selected to act as the return electrode for some or all sensing modes. The housing 40 may further be used as a return electrode alone or in combination with one or more of the electrodes of FIG. 1 for shocking purposes. The housing 40 further includes a connector (not shown) having a plurality of terminals 41-52. To achieve sensing, pacing and shocking in desired chambers of the heart, the terminals 41-52 are selectively connected to corresponding combinations of electrodes 22-38.

The IMD 10 includes a programmable microcontroller 60 that controls the various modes of sensing and stimulation therapy. The microcontroller 60 includes a microprocessor, or equivalent control circuitry, designed specifically for controlling sensing impedance derivation and the delivery of stimulation therapy and may further include RAM or ROM memory, logic and timing circuitry, state machine circuitry, and I/O circuitry. The microcontroller 60 includes the ability to process or monitor input signals (data) as controlled by a program code stored in memory. The details of the design and operation of the microcontroller 60 are not critical to the present invention. Rather, any suitable microcontroller 60 may be used.

The microcontroller 60 may search for a pacing threshold following paced events. The microcontroller 60 may do so, by performing an auto capture process to determine whether a paced event successfully captured the surrounding tissue. The microcontroller 60 includes an arrhythmia detection module 75 that analyzes sensed signals and determines when an arrhythmia (e.g., fibrillation) is occurring. The detection module 75 receives signals sensed by electrodes located at sensing sites. The detection module 75 detects arrhythmias, such as ventricular tachycardia (VT), bradycardia and ventricular fibrillation (VF). The microcontroller 60 may include a morphology detection module 73 that analyzes the morphology of the cardiac signal. Among other things, the module 73 may detect R wave peaks and/or detect T wave features of interest, such as onset, peak, etc.

An atrial pulse generator 70 and a ventricular pulse generator 72 generate pacing and ATP stimulation pulses for delivery by desired electrodes. The electrode configuration switch 74 (also referred to as switch bank 74) controls which terminals 41-52 receive shocks or pacing pulses. The atrial and ventricular pulse generators, 70 and 72, may include dedicated, independent pulse generators, multiplexed pulse generators, shared pulse generators or a single common pulse generator. The pulse generators 70 and 72 are controlled by the microcontroller 60 via appropriate control signals 76 and 78, respectively, to trigger or inhibit stimulation pulses. The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, etc.

An electrode configuration switch 74 connects the sensing electronics to the desired terminals 41-52 of corresponding sensing electrodes 22-38. For example, terminals 49-52 may be coupled to LV electrodes 23-26. The switch 74 may connect terminals 41-52 to one or more ventricular sensing circuits 84, which provide cardiac signals, representative of cardiac activity, to the microcontroller. The circuit 84 may amplify, filter, digitize and/or otherwise process the sensed cardiac signals from the LV electrodes 23-26. The circuit 84 may provide separate, combined or difference signals to the microcontroller 60 representative of the sensed signals from the LV electrodes 23-26. The circuit 84 may also receive sensed signals from RV electrodes 32 and 34 through terminals 43 and 44. The atrial sensing circuit 82 is connected through the switch 74 terminals 42 and 45-46 to desired RA and/or LA electrodes 22 and 27-28 to sense RA and/or LA cardiac activity. The switch 74 also connects various combinations of the electrodes 22-38 to an impedance measurement circuit 112.

An impedance measuring circuit 112 collects multiple measured impedances between corresponding multiple combinations of electrodes 22-38. For example, the impedance measuring circuit 112 may collect a measured impedance for each or a subset of the active sensing vectors 151-155. Optionally, the impedance measuring circuit 112 may also monitor lead impedance during the acute and chronic phases for proper lead positioning or dislodgement; detects operable electrodes and automatically switches to an operable pair if dislodgement occurs; measures respiration or minute ventilation; measures thoracic impedance for determining shock thresholds; detects when the device has been implanted; measures stroke volume; and detects the opening of heart valves, etc.

The switch bank 74 includes a plurality of switches for connecting the desired electrodes to the appropriate I/O circuits, thereby providing complete electrode programmability. The switch 74, in response to a control signal 80 from the microcontroller 60, determines the polarity of the stimulation pulses (e.g., unipolar, bipolar, co-bipolar, etc.) by selectively closing the appropriate combination of switches (not specifically shown). Atrial sensing circuits 82 and ventricular sensing circuits 84 may also be selectively coupled to the right atrial lead 20, LV lead 21, and the RV lead 30, through the switch 74 for detecting the presence of cardiac activity in each of the four chambers of the heart. The switch 74 determines the “sensing polarity” of the cardiac signal by selectively closing the appropriate switches.

The outputs of the atrial and ventricular sensing circuits 82 and 84 are connected to the microcontroller 60 which, in turn, is able to trigger or inhibit the atrial and ventricular pulse generators 70 and 72, respectively. The sensing circuits 82 and 84, in turn, receive control signals over signal lines 86 and 88 from the microcontroller 60 for purposes of controlling the gain, threshold, the polarization charge removal circuitry (not shown), and the timing of any blocking circuitry (not shown) coupled to the inputs of the sensing circuits, 82 and 86.

Cardiac signals are also applied to the inputs of an analog-to-digital (ND) data acquisition system 90. The data acquisition system 90 is configured to acquire intracardiac electrogram signals, convert the raw analog data into a digital signal, and store the digital signals for later processing and/or telemetric transmission to an external IMD 102. The data acquisition system 90 samples cardiac signals across any pair of desired electrodes. The data acquisition system 90 may be coupled to the microcontroller 60, or other detection circuitry, for detecting an evoked response from the heart 12 in response to an applied stimulus, thereby aiding in the detection of “capture.” Capture occurs when an electrical stimulus applied to the heart is of sufficient energy to depolarize the cardiac tissue, thereby causing the heart muscle to contract.

The microcontroller 60 is further coupled to a memory 94 by a suitable data/address bus 96. The memory 94 stores programmable operating, impedance measurements, impedance derivation and therapy-related parameters used by the microcontroller 60. The operating and therapy-related parameters define, for example, pacing pulse amplitude, pulse duration, electrode polarity, rate, sensitivity, automatic features, arrhythmia detection criteria, and the amplitude, wave shape and vector of each stimulating pulse to be delivered to the patient's heart 12 within each respective tier of therapy.

The impedance derivation parameters may include information designating i) sensing electrodes to use to define active sensing vectors, ii) sets and subsets of sensing vectors to use to monitor various regions of the heart, iii) sets or subsets of active sensing vectors to combine to form each pseudo sensing vector, iv) weight valves to use with active sensing vectors to form each pseudo sensing vector, v) algorithms for how to mathematically combine active sensing vectors to form each pseudo sensing vector, and the like.

The operating and therapy-related parameters may be non-invasively programmed into the memory 94 through a telemetry circuit 100 in telemetric communication with the external IMD 102, such as a programmer, trans-telephonic transceiver, or a diagnostic system analyzer. The telemetry circuit 100 is activated by the microcontroller 60 by a control signal 106. The telemetry circuit 100 advantageously allows intracardiac electrograms and status information relating to the operation of the IMD 10 (as contained in the microcontroller 60 or memory 94) to be sent to the external IMD 102 through an established communication link 104.

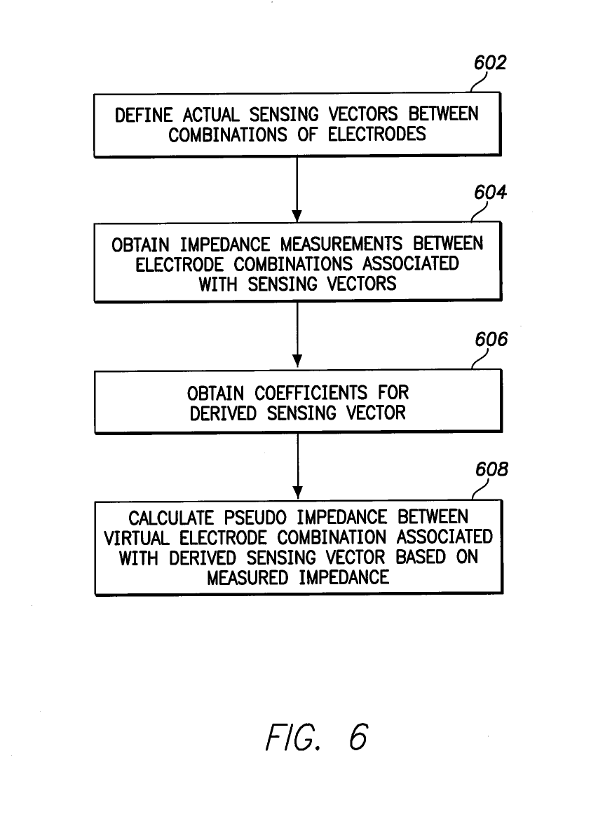

The microcontroller 60 includes an impedance derivation module 77 that derives impedances associated with pseudo sensing vectors based on impedance measurements along active sensing vectors. The impedance derivation module 77 performs the operations discussed herein in connection with FIG. 6.

The stimulation IMD 10 may include a physiologic sensor 108 to adjust pacing stimulation rate according to the exercise state of the patient. The physiological sensor 108 may further be used to detect changes in cardiac output, changes in the physiological condition of the heart, or diurnal changes in activity (e.g., detecting sleep and wake states). The battery 110 provides operating power to all of the circuits shown in FIG. 2.

The microcontroller 60 further controls a shocking circuit 116 by way of a control signal 118. The shocking circuit 116 generates stimulating pulses of low (up to 0.5 Joules), moderate (0.5-10 Joules), or high energy (11 to 40 Joules), as controlled by the microcontroller 60. Stimulating pulses are applied to the patient's heart 12 through at least two shocking electrodes, and as shown in this embodiment, selected from the left atrial (LA) coil electrode 28, the RV coil electrode 36, the SVC coil electrode 38 and/or the housing 40.

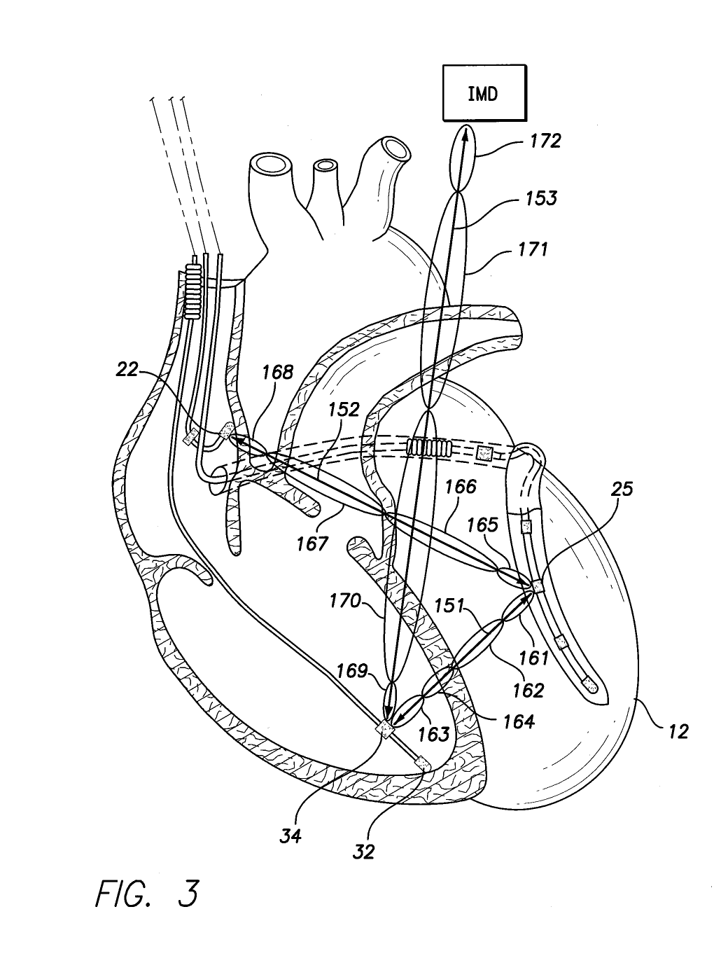

FIGS. 3-5 illustrate an expanded view of the heart 12 and portions of the leads 20, 21 and 30 from FIG. 1. FIGS. 3-5 will be discussed in connection with one example of determining which and how active sensing vectors should be combined. In FIGS. 3-5, the arrows and brackets showing the locations of the impedance components and the boundaries between the impedance components are only for illustrative purposes and not to be construed as limiting, nor to be interpreted as specific locations. The measured impedance along each active or pseudo sensing vector can be conceptually separated into impedance components. As shown in FIG. 3, the active sensing vector 151 is comprised of an LV internal component 161, an RV internal component 163, an LV path component 162 and an RV path component 164. The LV and RV internal components 161 and 163 represent impedance amounts due to the electrode impedance, the local tissue properties and the blood through which vector 151 passes within the LV and RV chambers immediately proximate the electrodes 34 and 25. The LV and RV path components 162 and 164 represent impedance amounts due primarily to the tissue and walls of the LV and RV chambers, respectively, and blood through which the vector 151 passes remote from the electrodes 34 and 25.

The vector 152 is comprised of LV and RA internal components 165 and 168, and LV and RA path components 166 and 167. The LV and RA internal components 165 and 168 are representative of the impedance of the electrodes, of the local tissue properties and the blood along the vector 152 in the LV and RA chambers immediately proximate to the electrodes 22 and 125. The LV and RA path components 166 and 167 are representative of the impedance of the blood, tissue, chamber walls, veins and aorta through which the vector 152 passes remote from the electrodes 22 and 25.

The vector 153 is comprised of an RV internal component 169, an RV path component 170, a CAN path component 171 and a CAN interface component 172. The RV internal component 169 is representative of the electrode impedance, of the local tissue properties and the impedance of the blood immediately proximate to the RV chamber along the vector 153. The CAN interface component 172 is representative of the impedance of the IMD 10 and due to the tissue, lungs and any other structure along the vector 153 outside the heart 12 and immediate proximate to the IMD 10. The CAN path component 171 is representative of the impedance due to the aortic region and any other structure along the vector 153 proximate the heart 12 and not included in the CAN interface component 172. The RV path component 170 is representative of the impedance due to blood, tissue and muscle of the RV chamber, but remote from the RV electrode 34. The RV path component 170 also includes the impedance due to the tissue, blood and muscle of the LV and LA chambers along the vector 153.

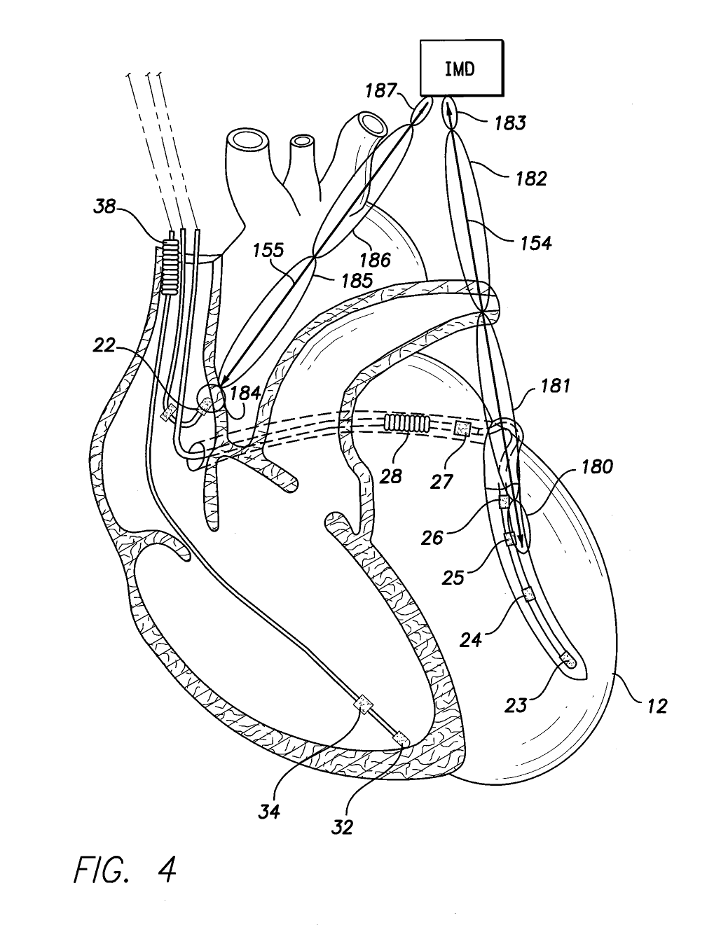

FIG. 4 illustrates the active sensing vectors 154 and 155. The vector 154 is comprised of an LV internal component 180, an LV path component 181, a CAN path component 182 and a CAN interface component 183. The LV internal component 180 is representative of the impedance at the LV electrode 25, and of the impedance value due to the local tissue properties and blood along the vector 154 within the LV chamber immediately proximate to the LV electrode 25. The LV path component 181 is representative of the impedance due to the LV tissue and the LV chamber wall and along the vector 154 as well as some of the tissue and muscle outside the heart along the vector 154. The CAN interface component 183 is representative of the impedance of the IMD CAN electrode and of the tissue and body structure immediately proximate to the IMD 10 along the vector 154. The CAN path component 182 is representative of the impedance value due to the tissue and aorta structure along the vector 154 and remote from the IMD 10.

The vector 155 is comprised of an RA internal component 184, an RA path component 185, a CAN path component 186 and a CAN interface component 187. The RA internal component 184 is representative of the impedance due to blood within the RA chamber of the impedance of the RA electrode 22 and the local tissue properties immediately proximate to the RA electrode 22. The RA path component 185 is representative of the impedance value due to the RA tissue and wall proximate to the RA chamber, but remote from the RA electrode 22, along the vector 155. The CAN path component 186 is representative of the impedance due to the tissue and aortic structure along the vector 155 remote from the IMD 10. The CAN interface component 187 is representative of the impedance of the IMD CAN electrode and of the impedance due to the tissue and body structure immediately proximate to the IMD 10 along the vector 155.

As explained below, by recognizing the various separate components of each active sensing vector, select combinations of the active sensing vectors 150-155 may be combined through weighting functions and summing operations to form other combinations of impedance components and to derive impedance estimates for pseudo sensing vectors that cannot be directly measured.

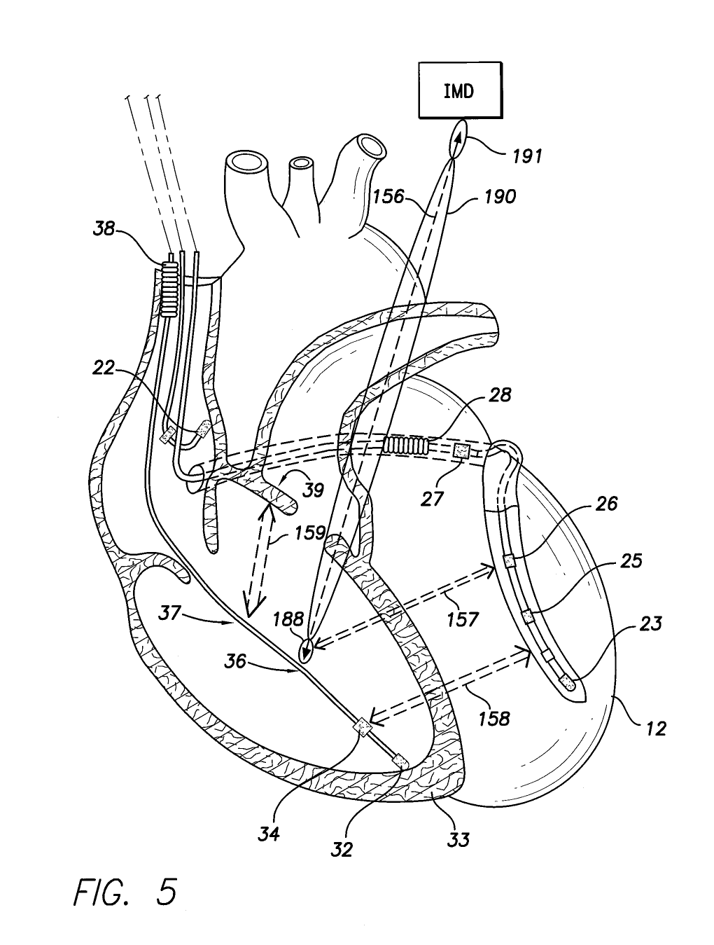

FIG. 5 illustrates examples of pseudo sensing vectors, such as the pseudo sensing vector 156 which extends from the IMD 10 to a pseudo sensing site 36 that is located at an intermediate point within the RV chamber. In the example of FIG. 5, pseudo sensing site 36 is located at a point where an RV coil electrode would normally be located when a shocking lead is implanted into the RA chamber. The pseudo sensing site 36 is located remote from the apex 33 of the RV chamber and spaced apart from the RV electrode 34.

The vector 156 is comprised of an RV internal component 188, a CAN path component 190 and a CAN interface component 191. The RV internal component 188 is representative of the impedance due to the blood within the RV chamber along the vector 156 immediately adjacent to the pseudo sensing site 36. The CAN path component 190 is also representative of impedance due to tissue and wall structure proximate the RV chamber along vector 156 remote from the pseudo sensing site 36. The CAN path component 190 is also representative of impedance due to tissue, blood and wall structures of the heart remote from the RV chamber and along vector 156. The CAN interface component 191 is representative of impedance due to the tissue and body structures immediately proximate to the CAN electrode of the IMD 10. The following equations summarize the above discussed impedance components that may be combined to form impedances along the vectors 149-156.

(LV Ring 25 to RV Ring 34)=LV(internal)+RV(internal)+Path(LV)+Path(RV)

(LV Ring 25 to RA Ring 22)=LV(internal)+RA(internal)+Path(LV)+Path(RA)

(RV Ring 34 to Can 10)=RV(internal)+Can(interface)+Path(RV)+Path(Can)

(LV Ring 25 to Can 10)=LV(internal)+Can(interface)+Path(LV)+Path(Can)

(RA Ring 22 to Can 10)=RA(internal)+Can(interface)+Path(RA)+Path(Can)

(RV coil 36 to Can 10)=RV_{coil}(internal)+Can(interface)+Path(Can)

The RV coil 36 can correspond to a real electrode in ICD or to a virtual sensing site 36 in a pacemaker where no active electrode is located. Based on the above equations, additional impedances may be estimated through derivation calculations. For example, certain combinations of the impedance components may be regrouped and transformed to derive different impedance groups. Sets of active sensing vectors may be combined to obtain impedance estimates for various portions of the heart. For example, the sensing vector 155 (RA ring to CAN impedance) may be summed with vector 152 (LV ring to RA ring impedance) and vector 154 (LV ring to CAN impedance) may be subtracted therefrom. This resulting value may then be divided by a constant (e.g., 2) to arrive at an estimate of a total impedance associated with the RA chamber (representing both the internal and path impedance for the right atrium). In addition, the vector 152 (LV ring to RV ring impedance) may be summed with vector 153 (RV ring to CAN impedance) and have subtracted therefrom the vector 154 representing the LV ring to CAN impedance. This resulting impedance may then be divided by a constant (e.g., 2) to arrive at an estimate of the total impedance associated with the RV chamber. The total impedance associated with the RV chamber includes both an RV internal component and an RV path component.

The vector 152 (LV ring to RA ring impedance) may be summed with vector 154 (LV ring to CAN impedance) and has subtracted therefrom vector 155 (RA ring to CAN impedance). This value would then be divided by a constant (e.g., 2) to arrive at the total impedance associated with the LV chamber. The total impedance associated with the LV chamber would include an LV internal component and an LV path component.

The vector 155 (RA ring to CAN impedance) may be summed with vector 154 (LV ring to CAN impedance) and have subtracted therefrom vector 152 (LV ring to RA ring impedance). The result would then be divided by a constant (e.g., 2) to arrive at an impedance representative of the “A&LV to CAN” impedance. The A&LV to CAN impedance corresponds to the impedance of the CAN interface component and the impedance of the CAN path component.

The vector 153 (RV ring to CAN impedance) may be summed with vector 154 (LV ring to CAN impedance) and have subtracted therefrom vector 152 (LV ring to RV ring impedance). This result would then be divided by a constant (e.g., 2) to arrive at the RV&LV to CAN impedance. The RV&LV to CAN impedance represents the combination of the impedance associated with the CAN interface and the impedance associated with the CAN path. In general, the CAN interface impedance would remain constant due to in part to the large surface area of the CAN interface. If the CAN interface impedance remains constant, then the A&LV to CAN impedance and the RV&LV to CAN impedance would have substantially equal values.

With reference to FIG. 5, as explained above, the pseudo sensing vector 156, which corresponds to the impedance between RV coil site 36 and CAN, is comprised of an RV internal component 188, a CAN path component 190 and a CAN interface component 191. The RV internal component 188 would generally remain substantially constant due in part to the large surface area of the interface.

The impedance associated with the vector between the RV coil and CAN can be represented as an approximation of the A&LV to CAN impedance plus a constant. Similarly, the impedance associated with the RV coil to CAN vector 156 equals the RV&LV to CAN impedance plus a constant which can be approximated by the vector between the RA ring and CAN when summed with the vector between the LV ring and CAN and subtracting the vector between the LV ring and RV ring divided by a constant (e.g., 2). This same impedance equals the impedance of the vector 153 between the RV ring and CAN plus the impedance from the vector 154 between the LV ring and CAN minus the impedance from the vector 152 between the LV ring and RV ring divided by a constant (e.g., 2). The above discussion of the interrelation between impedance associated with different vectors and components of vectors is illustrated in the following equations.

$$\begin{matrix}RA ( imp ) = ( \begin{matrix}( RA Ring to Can ) + ( LV Ring to RA Ring ) - \\ ( LV Ring to Can )\end{matrix} ) / 2 \\ = RA ( internal ) + Path ( RA )\end{matrix}$$

$$\begin{matrix}RV ( imp ) = ( \begin{matrix}( LV Ring to RV Ring ) + ( RV Ring to Can ) - \\ ( LV Ring to Can )\end{matrix} ) / 2 \\ = RV ( internal ) + Path ( RV )\end{matrix}$$

$$\begin{matrix}LV ( imp ) = ( \begin{matrix}( LV Ring to RA Ring ) + ( LV Ring to Can ) - \\ ( RA Ring to Can )\end{matrix} ) / 2 \\ = ( \begin{matrix}( LV Ring to RV Ring ) + ( LV Ring to Can ) - \\ to ( RV Ring to Can )\end{matrix} ) / 2 \\ = LV ( internal ) + Path ( LV )\end{matrix}$$

$$\begin{matrix}A \text{\&} LVtoCan ( imp ) = ( \begin{matrix}( RA Ring to Can ) + \\ ( LV Ring to Can ) - \\ ( LV Ring to RA Ring )\end{matrix} ) / 2 \\ = Can ( interface ) + Path ( Can )\end{matrix}$$

$$\begin{matrix}RV \text{\&} LVtoCan ( imp ) = ( \begin{matrix}( RV Ring to Can ) + \\ ( LV Ring to Can ) - \\ ( LV Ring to RV Ring )\end{matrix} ) / 2 \\ = Can ( interface ) + Path ( Can )\end{matrix}$$

The Can (interface) would be a constant, because of the large surface of the interface, leaving A&LVtoCan(imp) and RV&LVtoCan(imp) with equal values.

(RV coil to Can)=RV_{coil}(internal)+Can(interface)+Path(Can)

The RV_{coil} (internal) would be a constant, because of the large surface of the interface. The (RV coil to Can) impedance can be rewritten as follows:

$$\begin{matrix}≅ A \text{\&} LVtoCan ( imp ) + constant \\ = RV \text{\&} LVtoCan ( imp ) + constant \\ ≅ ( ( RA Ring to Can ) + ( LV Ring to Can ) - ( LV Ring to RA Ring ) ) / 2 \\ = ( ( RV Ring to Can ) + ( LV Ring to Can ) - ( LV Ring to RV Ring ) ) / 2\end{matrix}$$

Thus, the derived impedances from pacing lead can be used as aforementioned (A&LVtoCan(imp)+constant and RV&LVtoCan(imp)+constant) to substitute the RV_{coil} lead vector in the CRT-P system to provide better accuracy and specificity for the algorithm. The derived measurements can also be used for self-calibration purposes, such that where there is system error for the measurement, such as gain or offset.

FIG. 6 illustrates a processing sequence to be carried out in accordance with an embodiment of the present invention for estimating impedance associated with pseudo sensing vectors for an IMD, such as a pacemaker. Beginning at 602, the method defines or determines actual sensing vectors that exist between combinations of electrodes located on leads within or proximate to the heart. The defining or determining operation at 602 may be performed prior to implantation, during a programming operation by a physician post implantation, or automatically by the IMD after implantation.

At 604, the method obtains impedance measurements between the electrode combinations determined at 602. The electrode combinations are associated with the actual sensing vectors that are supported by the present types and configuration of leads implanted in the patient. At 604, multiple measured impedances are collected between corresponding multiple combinations of electrodes. Each combination of electrodes is associated with an active sensing vector. By way of example only, an individual active sensing vector may extend between a pair of electrodes. Alternatively, an active sensing vector may be defined through the use of three or more existing electrodes, such as when two or more electrodes are rendered electrically common with one another. For example, the measured impedances may be obtained by a pacemaker utilizing pacemaker leads where the measured impedances are taken along active pacemaker sensing vectors.

The collecting operation may utilize at least one electrode on at least one pacemaker lead to obtain a measured impedance. For example, the electrodes may include a right ventricular (RV) ring electrode, a left ventricular (LV) ring electrode. The electrodes may also utilize the housing of the IMD (referred to as the CAN). In the foregoing example, an active sensing vector extends between the RV ring and CAN electrodes. Another active sensing vector extends between the LV ring and RV ring electrodes and a third active sensing vector extends between the LV ring and the CAN electrodes. For example, the electrode combination may include an RA electrode 22, an LV electrode 25, and a CAN electrode at the IMD 10. The active sensing vectors would include 152 between the LV and RA electrodes 25 and 22, active sensing vector 154 between the LV electrode 25 and CAN, and active sensing vector 153 between the RV electrode 34 and the CAN.

At 606, the method obtains coefficients associated with derived sensing vectors that are to be calculated. The coefficients may be stored in memory in the IMD, programmed by a physician through an external programmer, automatically determined by the IMD during operation and the like.

At 608, the derived impedance is calculated for a virtual electrode combination that is associated with a pseudo sensing vector. The derived impedance is calculated based on the measured impedances and the coefficients that are obtained for the derived sensing vector.

The pseudo sensing vector extends to or from at least one pseudo sensing site. The pseudo sensing vector may extend to or from an active sensing site corresponding to an active electrode as well. The calculation may include determining the derived impedance for a non-pacemaker sensing vector, such as a sensing vector associated with a shocking coil. The non-active sensing site may correspond to a virtual electrode location that does not include an active sensing electrode. For example, the pseudo sensing site may correspond to an intermediate location within the right ventricle, at which no actual electrode is positioned. For example, the pseudo sensing vector 156 (FIG. 5) extends to and from the CAN of the IMD 10 which represents an actual electrode and an active sensing site. The pseudo sensing vector 156 also extends to and from a virtual sensing site 36, at which no actual electrode is configured to perform active sensing. It should be recognized that a shocking coil or other shocking or stimulus delivery electrodes may be located at the virtual sensing site 36. However, such a shocking electrode may not be utilized during a sensing operation.

Optionally, the virtual sensing vector may extend between 2 or more virtual sensing sites. For example, the derived impedances may be determined for one or more alternative pseudo sensing vectors, such as illustrated in FIG. 5 at vector 157, 158 and 159. By way of example only, vector 157 extends from an active sensing site at electrode 26 to a pseudo sensing site 36. Pseudo sensing vector 158 extends between an active sensing site at RV electrode 34 to a pseudo sensing site in the left ventricle. Pseudo sensing vector 159 extends between pseudo sensing sites generally denoted at 37 and 39. Pseudo sensing site 37 is located at an upper intermediate region with the right ventricle, while pseudo sensing site 39 is located proximate a wall between the right and left atrium and near the aortic vessels.

The calculating operation may utilize the weighting coefficients to weight one or more of the measured impedances, thereby obtaining a weighted impedance measurement. The weighted impedance measurements may be then summed and optionally normalized or averaged to obtain the derived impedance. By way of example, the pseudo sensing vector may represent a shock coil impedance vector that extends to or from a pseudo shock coil sensing site that is not an actual active sensing site.

In accordance with the methods and systems described above, derived impedance values are estimated along desired vectors through the heart. The foregoing methods and systems allow the derivation of impedances based upon various combinations of active sensing sites, thereby providing greater flexibility in lead and electrode configurations and types to be used with an IMD in connection with various algorithms for monitoring and diagnosing heart conditions.

The impedance for the pseudo sensor vector may be calculated using only a subset of the available actual sensing vectors. For example, the impedance for the pseudo sensing vector 156 may be calculated based only on measured impedances along actual sensing vectors 152, 155 and 156. Alternatively, the impedance for the pseudo sensing vector 156 may be calculated based only on measured impedances along the actual sensing vectors 152, 153 and 154.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for estimating an impedance associated with a pseudo sensing vector for an implantable medical device (IMD), comprising:
collecting multiple measured impedances between corresponding multiple combinations of electrodes, each combination of electrodes being associated with an active sensing vector; and
calculating a derived impedance for at least one pseudo sensing vector based on the measured impedances, wherein the pseudo sensing vector extends to or from at least one pseudo sensing site.

2. The method of claim 1, wherein the collecting includes obtaining measured impedances by a pacemaker along active pacemaker sensing vectors and the calculating determines the derived impedance for a non-pacemaker sensing vector.

3. The method of claim 1, wherein the non-active sensing site corresponds to a virtual electrode location that does not include an active sensing electrode.

4. The method of claim 1, further comprising obtaining weighting coefficients associated with the active sensing vectors, the calculating including calculating the derived impedance based on the weighting coefficients.

5. The method of claim 1, wherein the calculating includes weighting the measured impedances to obtain weighted impedance measurements and summing the weighted impedance measurements.

6. The method of claim 1, wherein the pseudo sensing vector represents a shock-coil impedance vector that extends to or from at least one pseudo shock-coil sensing site that is a non-sensing site.

7. The method of claim 1, wherein the collecting includes utilizing at least one electrode on at least one pacemaker lead to obtain the measured impedances.

8. The method of claim 1, wherein the electrodes include a right ventricular (RV) ring electrode, a left ventricular (LV) ring electrode and a housing of the IMD (CAN), the active sensing vectors extend between i) the RV ring and CAN electrodes, ii) the LV ring and RV ring electrodes and iii) the LV ring and CAN electrodes.

9. The method of claim 1, wherein the pseudo sensing site corresponds to an intermediate location within the right ventricle (RV).

10. An implantable medical device (IMD), comprising:
inputs configured to be coupled to leads having electrodes thereon, wherein combinations of the electrodes are associated with respective active sensing vector;
impedance measurement module to collect multiple measured impedances between corresponding combinations of the electrodes; and
impedance derivation module to calculate a derived impedance for at least one pseudo sensing vector based on the measured impedances, wherein the pseudo sensing vector extends to or from at least one pseudo sensing site.

11. The IMD of claim 10, wherein the IMD constitutes a pacemaker and the impedance measurement module obtains measured impedances along active pacemaker sensing vectors, the impedance derivation module determines the derived impedance for a non-pacemaker sensing vector.

12. The IMD of claim 10, wherein the impedance derivation module calculates the derived impedance for a non-active sensing site that corresponds to a virtual electrode location that does not include an active sensing electrode.

13. The IMD of claim 10, wherein the impedance derivation module obtains weighting coefficients associated with the active sensing vectors and calculates the derived impedance based on the weighting coefficients.

14. The IMD of claim 10, wherein the impedance derivation module weights the measured impedances to obtain weighted impedance measurements and sums the weighted impedance measurements.

15. The IMD of claim 10, wherein the pseudo sensing vector represents a shock-coil impedance vector that extends to or from at least one pseudo shock-coil sensing site that is a non-sensing site.

16. The IMD of claim 10, further comprising a pacemaker lead with pacemaker electrodes thereon, the inputs being connected to the pacemaker electrodes on the pacemaker lead to obtain the measured impedances.

17. The IMD of claim 10, further comprising leads with a right ventricular (RV) ring electrode and a left ventricular (LV) ring electrode, the IMD having an active housing (CAN), the active sensing vectors extend between i) the RV ring and CAN electrodes, ii) the LV ring and RV ring electrodes and iii) the LV ring and CAN electrodes.

18. The IMD of claim 10, wherein the pseudo sensing site corresponds to an intermediate location within the right ventricle (RV).
